(19) United States
(12) Patent Application Publication
(10) Pub. No.: US 2022/0012644 A1
(43) Pub. Date: Jan. 13, 2022

(54) METHOD AND SYSTEM FOR MONITORING A REMOTE SYSTEM

(71) Applicant: Oxford University Innovation Limited, Oxford (Oxfordshire) (GB)

(72) Inventors: David CLIFTON, Oxford (Oxfordshire) (GB); Patrick THOMPSON, Oxford (Oxfordshire) (GB); Heloise GREEFF, Oxford (Oxfordshire) (GB); Achut MANANDHAR, London (Greater London) (GB)

(21) Appl. No.: 17/296,302

(22) PCT Filed: Oct. 21, 2019
(86) PCT No.: PCT/GB2019/052999

§ 371 Date: May 24, 2021

(30) Foreign Application Priority Data
Dec. 3, 2018 (GB) 1819717.8

(51) Int. Cl. G06N 20/20; G05B 23/02; G16H 50/30; F04B 9/14

(52) U.S. Cl. CPC G06N 20/20; G05B 23/0235; F04B 2201/12; F04B 9/14; G16H 50/30

(57) ABSTRACT

This disclosure relates to methods and apparatus for monitoring a remote system. In one arrangement, a plurality of measurement data units are obtained. Each measurement data unit represents a time series of measurements made by a sensor system at the remote system. A first trained machine learning model is used to identify a subset of the measurement data units that have a higher average probability of corresponding to an abnormal state of the remote system than the other measurement data units. Data representing the identified measurement data units is sent over a communications network to a central data processing system. An abnormal state of the remote system is detected by using a second trained machine learning model at the central data processing system to process the data representing the identified measurement data units.

The invention relates to monitoring a remote system, particularly for the purpose of detecting or predicting a transition of the remote system from a normal state to an abnormal state. The method is particularly applicable to monitoring components of rural infrastructure such as hand-operated water pumps.

Rural infrastructure, such as hand-operated pumps, play an important role in improving quality of life and driving economic growth, particularly in developing countries. Sustainable provision of reliable infrastructure requires a high standard of both installation and maintenance, but under-investment or neglect is widespread. Downtime due to system failure in rural settings is often greater than in urban settings due to practical challenges in the supply of spare parts combined with a lack of local skills. In sub-Saharan Africa, it is estimated that around one third of the one million hand-operated pumps used daily by nearly 200 million people are not working at any time and often remain broken for up to 30 days.

Predictive health monitoring is widely used in engineering applications to detect damage to infrastructure at an early stage. Forecasting failure rather than merely detecting failure once it occurs helps to reduce the downtime of systems, and, ideally, performing predictive maintenance can avoid downtime completely. With this approach already widely used in many fields from commercial and military jet engines, through to patient monitoring in health systems, it has also been used to monitor the condition and use of rural infrastructure.

V. Mehra, R. Ram, and C. Vergara, “A novel application of machine learning techniques for activity-based load disaggregation in rural off-grid, isolated solar systems,” GHTC 2016—IEEE Global Humanitarian Technology Conference: Technology for the Benefit of Humanity, Conference Proceedings, pp. 372-378,2016 discloses application of predictive health monitoring to off-grid solar home systems.

The following publications discloses application of predictive health monitoring to hand-operated pumps:

P. Thomson, R. Hope, and T. Foster, “Is silence golden? Of mobiles, monitoring, and rural water supplies,” Waterlines, vol. 31, no. 4, pp. 280-292, 2012;

F. Colchester, H. Greeff, P. Thomson, R. Hope, and D. Clifton, “Smart Hand-operated pumps: A Preliminary Data Analysis,” in Appropriate Healthcare Technologies for Low Resource Settings, 2014, pp. 1-4;

C. Nagel, J. Beach, C. Iribagiza, and E. A. Thomas, “Evaluating Cellular Instrumentation on Rural Hand-operated pumps to Improve Service Delivery-A Longitudinal Study in Rural Rwanda,” Environmental Science and Technology, vol. 49, no. 24, pp. 14 292-14 300, 2015;

E. Thomas, Z. Zumr, J. Graf, C. Wick, J. McCellan, Z. Imam, C. Barstow, K. Spiller, and M. Fleming, “Remotely Accessible Instrumented Monitoring of Global Development Programs: Technology Development and Validation,” Sustainability, vol. 5, no. 8, pp. 3288-3301, 2013;

P. Thomson, R. Hope, and T. Foster, “GSM-enabled remote monitoring of rural hand-operated pumps: a proof-of-concept study,” Journal of Hydroinformatics, vol. 14, no. 4, p. 829, October 2012.

Despite the importance of rural infrastructure and the potential impact of predictive monitoring, the implementation of remote condition monitoring systems in these extreme rural settings has historically been limited to data loggers. This is largely due to the technical and logistical challenges, such as battery life, data-transmission bandwidth limitations and long or expensive maintenance cycles, associated with operating in such remote locations. This necessitates the use of sensors that are robust, reliable, low-power and low-cost. These constraints can compromise performance, leading to data that is lower frequency, more coarsely quantised or with a poor signal-to-noise ratio.

D. L. Wilson, J. R. Coyle, and E. A. Thomas, “Ensemble machine learning and forecasting can achieve 99% uptime for rural hand-operated pumps,” PLoS ONE, vol. 12, no. 11, pp. 1-13, 2017 discloses that the use of ensemble machine learning in remote monitoring of rural hand-operated pumps, when combined with a preventive maintenance service model, could increase the uptime in rural hand-operated pumps to 99 percent. Although such performance improvements can translate directly into positive health impacts for local communities, the proposed model sacrifices prediction sensitivity (51.0%) over specificity (99.3%) when identifying independent failure events. However, when considering the failures as a series of “failure days”, the proposed method correctly identifies 24 out of 25 events. Since failures are novel events, a low sensitivity would lead to a high number of false alerts, which is undesirable in the context of rural monitoring due to the cost required to follow up each alert.

It is an object of the invention to at least partly address one or more of the issues described above.

According to an aspect, there is provided a method of monitoring a remote system, comprising: obtaining a plurality of measurement data units, each measurement data unit representing a time series of measurements made by a sensor system at the remote system; using a first trained machine learning model to identify a subset of the measurement data units that have a higher average probability of corresponding to an abnormal state of the remote system than the other measurement data units; sending data representing the identified measurement data units over a communications network to a central data processing system; and detecting an abnormal state of the remote system by using a second trained machine learning model at the central data processing system to process the data representing the identified measurement data units.

Thus, a method is provided which uses a combination of a first trained machine learning model running locally at a remote system and a second trained machine learning model running at a central data processing system that is connected to the remote system, at least intermittently, by a communications network. The first trained machine learning model operates effectively as a filter to identify measurement data that is more likely to contain information relevant to detecting an abnormal state of the remote system, allowing only that data to be stored and/or transmitted to the central data processing system. The inventors have found that this filtering functionality can be achieved using very lightweight data processing hardware at the remote system without excessively compromising the ability of the method as a whole to reliably detect abnormal states of the remote system. The approach thus makes it possible to monitor remote systems effectively even in locations where computer processing power and/or data transmission capabilities are highly restricted, such as in remote rural locations in developing countries. The methodology is furthermore demonstrated to provide high sensitivity, thereby reducing the occurrence of costly false alarms.

In an embodiment, the first trained machine learning model estimates a probability of the remote system being in the abnormal state during a time period corresponding to each measurement data unit and the identification of the subset of measurement data units comprises identifying measurement data units corresponding to time periods in which the estimated probability is above a predetermined threshold. In an embodiment, the first trained machine learning model uses logistic regression to estimate the probabilities. Logistic regression has been found to be particularly well suited to implementing the first trained machine learning model, achieving a desirable balance between performance and data processing requirements.

In an embodiment, the remote system comprises a mechanical apparatus and the sensor system comprises an accelerometer. It has been found that the methodology works particularly efficiently when applied to mechanical apparatuses via accelerometry data. The methodology has been found to be particularly effective when applied to monitoring remotely installed hand-operated water pumps.

In an embodiment, the sensor system comprises an accelerometer configured to measure a component of acceleration of the handle parallel to a longitudinal axis of the handle. It has been found that a high proportion of information relevant to abnormality of a hand-operated pump is present in vibrations oriented longitudinally along the handle. Aligning an accelerometer with this axis and using the output as the basis both for the identification of the subset of measurement data units most relevant to abnormality and for the detection of the abnormal state by the second trained machine learning model promotes both efficient implementation (e.g. requiring minimal hardware, power and data transmission at the pump) and high sensitivity.

In an embodiment, the method further comprises pre-processing the measurement data units before the measurement data units are used by the first trained machine model, wherein the pre-processing comprises determining a period of a largest periodic component of the times series of measurements in each measurement data unit, and the pre-processing comprises removing measurement data units in which a period of the determined largest periodic component is below a predetermined threshold period, and wherein the remote system comprises a hand-operated water pump and the predetermined threshold period equals 0.5s. This approach efficiently avoids transmission of relatively low quality data from the remote system to the central data processing unit, for example data associated with children playing with pumps rather than using them properly.

In an embodiment, the pre-processing comprises applying a high pass filter to each measurement data unit. This approach has been found to improve an average quality of data transmitted from the remote system to the central data processing system by avoiding sending of data representing relatively low frequency movements associated directly with manual operation of the remote system, which contain a relatively low proportion of information correlated with abnormality in the state of the remote system.

According to an alternative aspect, there is provided a system for monitoring a remote system, comprising: a local data acquisition unit comprising a sensor system and a local data processing unit; and a central data processing system; wherein: the local data processing unit is configured to: obtain a plurality of measurement data units, each measurement data unit representing a time series of measurements made by the sensor system at the remote system; use a first trained machine learning model to identify a subset of the measurement data units that have a higher average probability of corresponding to an abnormal state of the remote system than the other measurement data units; and send data representing the identified measurement data units over a communications network to the central data processing system; and the central data processing system is configured to: detect an abnormal state of the remote system by using a second trained machine learning model to process the data representing the identified measurement data units received from the local data acquisition unit.

Embodiments of the invention will now be described, by way of example only, with reference to the accompanying drawings in which corresponding reference symbols indicate corresponding parts, and in which:

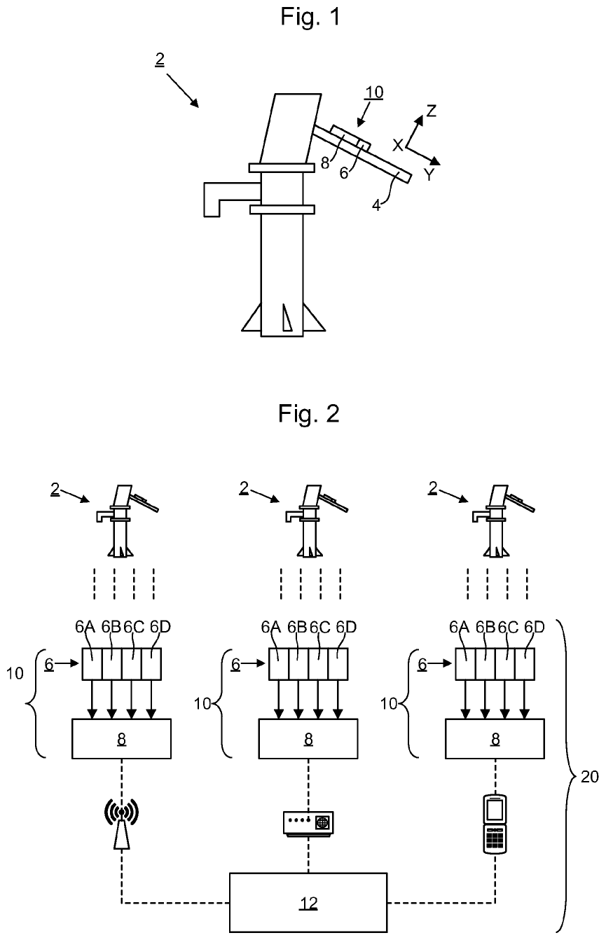

FIG. 1 depicts a remote system comprising a hand-operated pump and a local data acquisition unit attached to a handle of the pump;

FIG. 2 depicts a system for monitoring a plurality of remote systems;

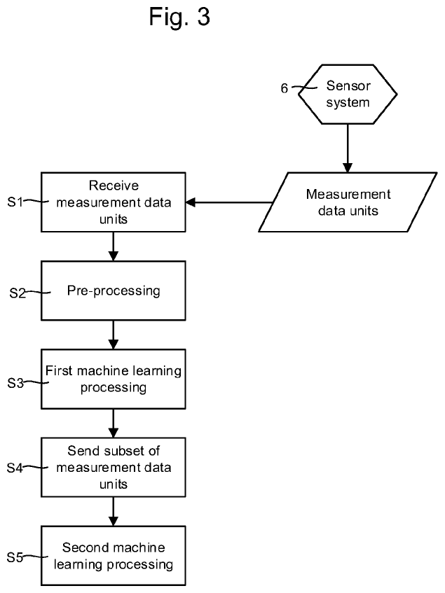

FIG. 3 is a flow chart depicting steps in a method of monitoring a remote system;

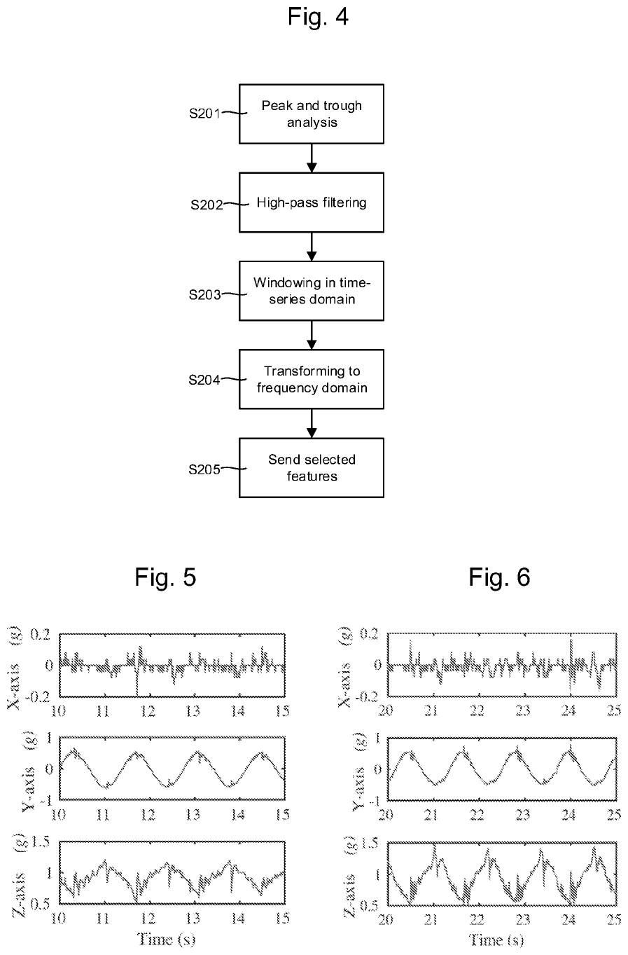

FIG. 4 is a flow chart depicting pre-processing steps to be applied to measurement data units prior to use of a first trained machine learning model to identify a subset of the measurement data units;

FIGS. 5 and 6 are graphs showing measurements from an accelerometer within a 5 s interval measuring operation of a hand-operated pump in a normal (FIG. 5) and abnormal (FIG. 6) condition, in each of the X, Y, and Z directions depicted in FIG. 1 (upper to lower plots, respectively, in each figure);

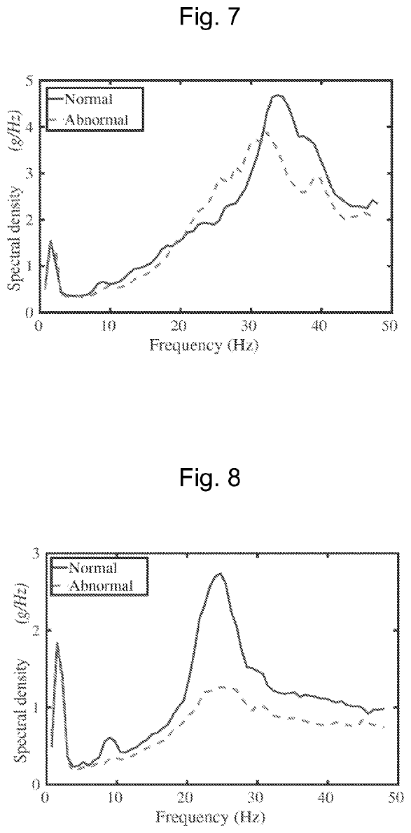

FIGS. 7 and 8 are graphs depicting the median amplitude of spectral data obtained by pre-processing measurement data units from hand-operated pumps respectively where the water is located at a relatively deep level (FIG. 7), greater than 25 m depth, and at a relatively shallow level (FIG. 8), less than 25 m depth;

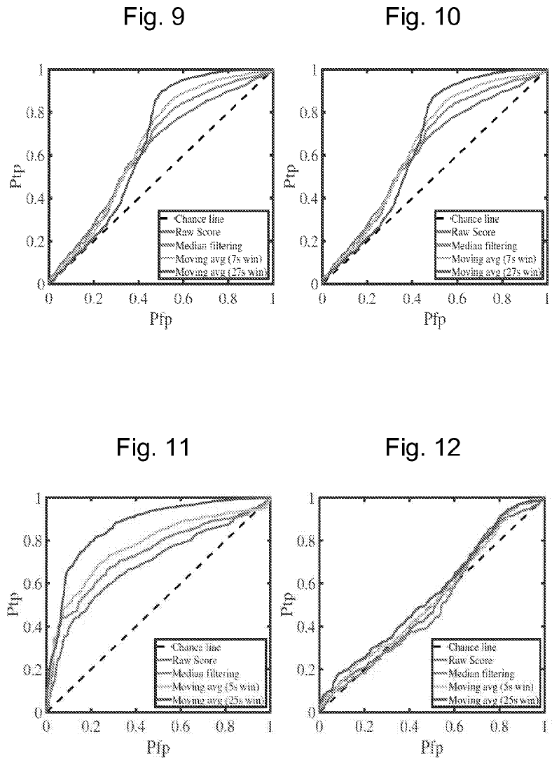

FIGS. 9-12 are graphs comparing the receiver operator curve (ROC) scores for a first machine learning model trained using different data subsets for: a) a general classifier trained using a first data set Dₘ (FIG. 9); b) a depth-specific inter-handpump classifier trained using a second data set D_{d,1} (FIG. 10); c) a broken pump rod in a depth-specific intra-handpump classifier trained using a second data set D_{d,2} (FIG. 11); d) a rising main leak in a depth-specific intra-handpump classifier trained using D_{d,2} (FIG. 12);

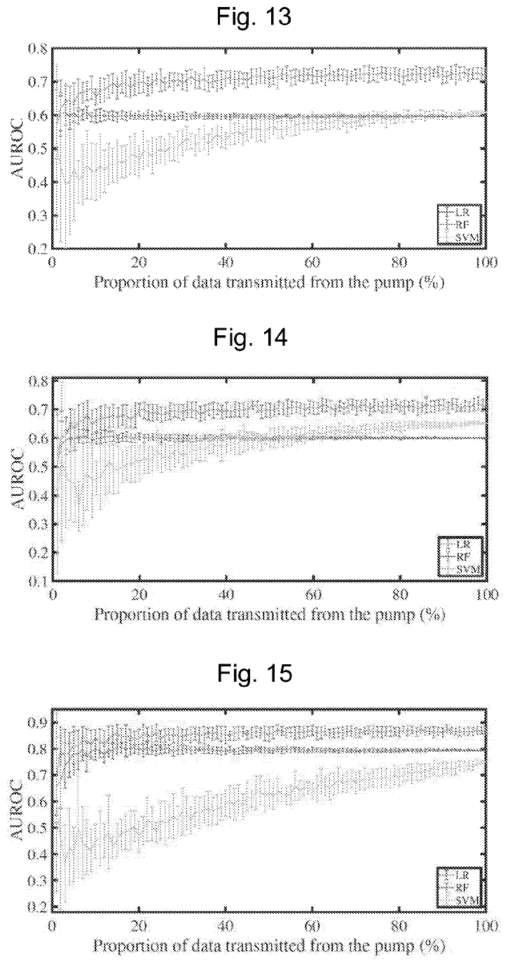

FIGS. 13-15 are graphs showing AUROC comparison of a second trained machine learning model using data representing measurement data units identified by the first trained machine learning model for: a general classifier trained using Dₘ (FIG. 13); a depth-specific inter-handpump classifier trained using D_{d,1} (FIG. 14); and a depth-specific intra-handpump classifier trained using D_{d,2} (FIG. 15);

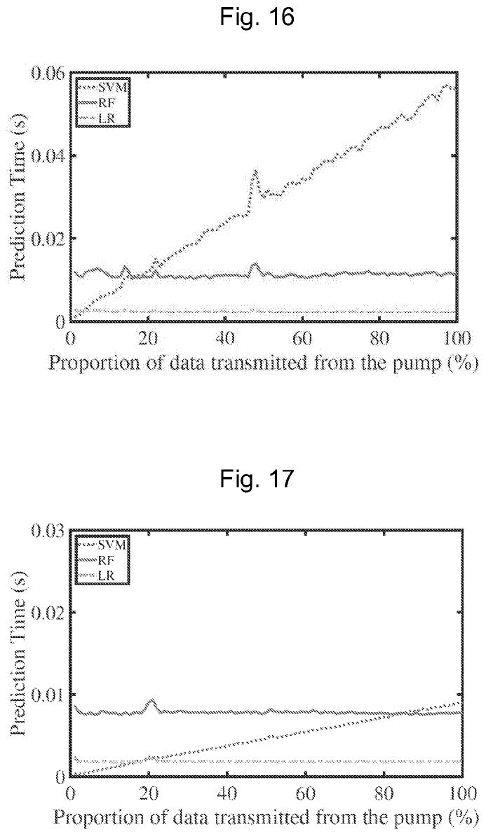

FIGS. 16-17 show comparison of run times for predictions by the second trained machine learning model as the proportion of data transmitted from the remote system to the second trained machine learning model varies for: an inter-hand-pump monitoring system (FIG. 16) and intra-hand-pump monitoring system (FIG. 17);

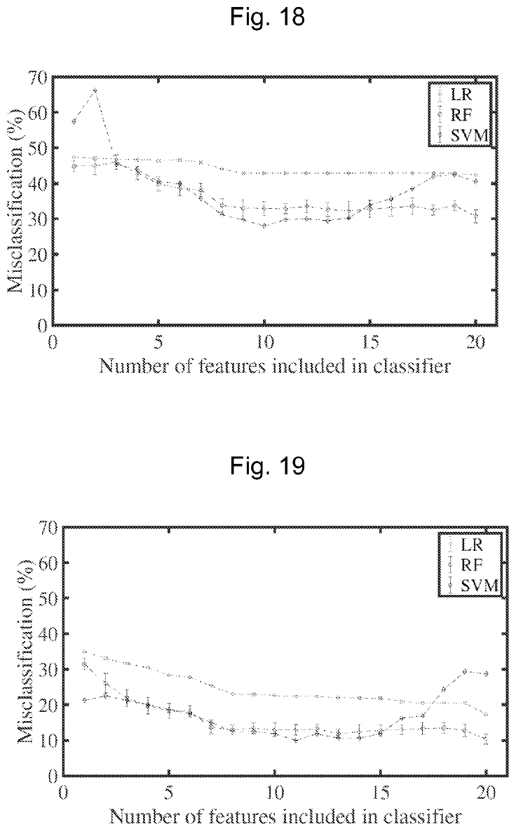

FIGS. 18-19 show comparison of the monitoring performance for varying numbers of features input to the classifying machine learning models applied to two deep well data sets for: an inter-hand-pump monitoring system (FIG. 18) and an intra-hand-pump monitoring system (FIG. 19).

The present disclosure relates to methods and systems for monitoring a remote system, such as an apparatus that is installed in a location where infrastructure such as high-speed internet and reliable power supplies are not readily available. Embodiments described below are particularly applicable to cases where the remote system comprises a mechanical apparatus having at least one moving part, such as a hand-operated water pump, but the principle may also be applied in other scenarios, including for remote monitoring of electrical infrastructure systems that are disconnected from the mains, such as off grid solar batteries, as well as for monitoring biological systems such as humans or animals in remote areas or using minimalist hardware or energy consumption.

FIG. 1 schematically depicts a hand-operated water pump 2, which is an example of a remote system 2 to which embodiments described below are applicable. The pump 2 comprises a movable handle 4 for hand-operating the pump 2 to cause water to be pumped out of a spout.

A sensor system 6 is provided for measuring physical characteristics associated with the remote system 2. In some embodiments, the sensor system 6 comprises an accelerometer attached to the remote system, for example to a moving part 4 of the remote system 2 or to another part which is affected by operation of the remote system (e.g. due to vibrations from operation propagating to that part).

In an embodiment, the sensor system 6 is provided as part of a local data acquisition unit 10. The local data acquisition unit 10 comprises the sensor system 6 and a local data processing unit 8. As will be described in detail below, the sensor system 6 generates measurement data units by making measurements at the remote system 2 and the local data processing unit 8 processes the measurement data units and sends data derived from the measurement data units over a communications network.

FIG. 2 depicts a system 20 for implementing methods of monitoring a remote system 2. In the example shown, the system 20 is shown as monitoring three remote systems 2 consisting of hand-operated water pumps, but the system could be configured to monitor many more remote systems, such as 10s are 100s of remote systems 2, as well as different types of remote systems. The system 20 comprises at least one of the local data acquisition units 10 for each of the remote systems 2 being monitored and a central data processing system 12.

In the embodiment shown, each sensor system 6 comprises multiple sensor elements 6A-6D. Each sensor element 6A-6D may obtain a different item of measurement information, such as acceleration data relative to a different one of plural axes, or measurement information concerning characteristics of the environment around the remote system 2, such as temperature, humidity, or rainfall data. In an embodiment where the remote system 2 comprises an electrical infrastructure system such as an off grid solar battery, the sensor system 6 may comprise a power meter configured to measure one or more characteristics of an electrical output from the electrical infrastructure system. In an embodiment where the remote system 2 comprises a biological system such as a human or animal, the sensor system 6 may obtain one or more of the following: heart rate, respiratory rate, temperature, blood oxygenation, systolic blood pressure, diastolic blood pressure, electrocardiogram, blood glucose, temperature, blood constituent levels, pupil size, pain score, Glasgow coma score, and/or analyze a sample from the human or animal.

FIG. 3 depicts a method of monitoring a remote system 2 using a system 20 such as that depicted in FIG. 2. Steps in the method are computer-implemented. The computer, which may be located either at the remote system 2 or at the central data processing system 12 depending on the step in question, may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media, optionally non-transitory media, storing computer readable instructions. When the computer readable instructions are read by the computer, the computer performs the required method steps. The computing hardware implementing the local data acquisition units 10 will typically be highly rudimentary (having much lower data processing capacity and speed, and lower power requirements) in comparison with the computing hardware provided at the central data processing system 12.

In step S1, a plurality of measurement data units are obtained from a sensor system 6 at a remote system 2. Each measurement data unit comprises a time series of measurements made by the sensor system 6. The time series of measurements may be univariate (e.g. where the sensor system 6 comprises a single sensor element) or multivariate (e.g. where the sensor system 6 comprises multiple sensor elements as in the example of FIG. 2). The system 20 may manage one remote system 2 or multiple remote systems f any number (e.g. 10s to 100s of remote systems 2 or more, depending on the type of system being monitoring and how densely they are provided in the region being managed).

In one specific embodiment, each local data acquisition unit 10 comprises a sensor system 6 comprising an IC-based, 96 Hz accelerometer, an 8-bit microprocessor, and a GSM modem. The estimated power consumption of this implementation of the local data acquisition unit 10 in a run and a sleep mode is compared in Table I.

TABLE I
Estimated power consumption of components of
an example local data acquisition unit 10 embedded
at a remote system operating at 25° C.

| | | |
|---|---|---|
| Component | Run Mode | Sleep Mode |

| | | | | |
|---|---|---|---|---|
| Microprocessor (8-bit at 8 MHz) | 20 | mA | 5 | mA |
| Accelerometer at 96 Hz | 200 | μA | 200 | μA |
| GSM Module (for SMS) | 250 | mA | 40 | μA |

As indicated schematically in FIG. 2, the communication network between the local data acquisition units 10 and the central data processing system 12 may be different for different ones of the remote systems 2, depending on what is available at each location. The capabilities of the local data acquisition units 10 may thus vary from one remote system 2 to another, being configured for example to communicate using a GSM modem in one instance, via WiFi in another instance, and/or by a mobile phone network connection in another instance.

In step S2, the measurement data units are pre-processed before being provided to the step S3 where the measurement data units will be processed by a first trained machine learning model.

FIG. 4 depicts an example pre-processing pathway suitable for use particularly in the context of monitoring hand-operated pumps.

In this example, the pre-processing comprises a step S201 based on peak and trough detection. In an embodiment, the peak and trough detection is used to determine a period of a largest (e.g. largest amplitude) periodic component of the times series of measurements in each measurement data unit. The pre-processing then removes measurement data units in which a period of the largest periodic component is below a predetermined threshold period. In the specific case of hand-operated pump monitoring, for example, it has been found beneficial to remove measurement data units corresponding to time periods in which the period of the largest periodic component is less than 0.5 s. This eliminates contributions that are less likely to be informative about the state of the remote system, for example because the contributions correspond to children playing on a hand-operated pump rather than the pump being used in the intended way.

In an embodiment, the pre-processing further comprises a step S202 comprising applying a high pass filter to each measurement data unit. With wishing to be bound by theory, the inventors believe this filtering is beneficial because changes in the underlying condition of the remote system 2 being monitored (e.g. a hand-operated pump) are not affected to a large extent by the relatively low frequency motion imparted to the moving part 5 (e.g. the handle of the hand-operated pump) directly by manual interaction by the user. In the context of the hand-operated pump, the high-pass filtering thus removes low-frequency components associated with the manual pumping tempo that are not strongly indicative of deterioration of the pump, while retaining information about fast-moving components such as vibrations.

In an embodiment, the pre-processing further comprises a step S203 comprising applying windowing in the time series domain. This may be used due to resource limitations at the local data acquisition unit 10, such as an 8-bit microprocessor and limited battery. In an embodiment, a phase-corrected 4-point moving average (MA) finite impulse response (FIR) filter to represent the shape of the recording, which is then removed from the original signal. The filter calculates the average of a number of points from the input signal such that each point of the output signal, y, is calculated as follows:

$$y [ i ] = \frac{1}{M} {\sum }_{j = 0}^{M - 1} x [ i + j ]$$

where x is the input signal and M is the number of points used in the moving average.

In an embodiment, the pre-processing further comprises a step S204 comprising transforming the measurement data units to represent the time series of measurements in the frequency domain. In an embodiment, Fast-Fourier transforms (FFTs) are used to decompose the signal into a sum of sinusoidal basis functions used to describe the frequency content within the time-series waveform. In one particular implementation, the recorded measurement data units were partitioned into 1.3 s windows with 50% overlap. This creates 128 samples per window, equivalent to 64 frequency components with a resolution of 0.75 Hz per component for a sampling frequency of 96 Hz. To account for truncated waveforms with discontinuous endpoints resulting from the finite windows, a 128-point Hamming window function was applied. The final result after such FFT application is a feature vector with 64 frequency components per window, up to the Nyquist rate.

In an embodiment, the pre-processing further comprises a step S205 in which selected features from the frequency domain representation provided by step S204 are output from the pre-processing. In one particular implementation, a subset of 20 features was selected by uniformly sampling across frequency bins 3 to 60, discarding low frequency components, equivalent to 0 to 2.25 Hz, which represent the pumping motion of the user, where a full hand-operated pump stroke has a median period of 1.1 s. Artefacts of this pumping motion can be seen in FIGS. 7 and 8, which depict the median amplitude of spectral data obtained by pre-processing measurement data units from hand-operated pumps respectively where the water is located at a relatively deep level (FIG. 7), greater than 25 m depth, and at a relatively shallow level (FIG. 8), less than 25 m depth.

In step S3 of FIG. 3, data representing the measurement data units output from the pre-processing of step S2 (i.e. pre-processed versions of the measurement data units) are provided to a first trained machine learning model. The first trained machine learning model identifies a subset of the measurement data units that have a higher average probability of corresponding to an abnormal state of the remote system than the other measurement data units. This functionality may be referred to as novelty filtering. The first machine learning model is a lightweight machine learning model in comparison to a second machine learning model (described below) due to the need for the first machine learning model to operate on the local data acquisition units 10. As will be demonstrated below, however, this early machine learning based identification of the most relevant measurement data units still makes it possible to greatly reduce the amount of data that is transmitted by the local data acquisition units 10 while allowing the remote systems to be monitored effectively. Downtime of the remote systems 2 can be avoided or reduced (e.g. by providing maintenance before failure) without require large expenditure on sophisticated local data acquisition units 10 and/or communications or power infrastructure to support them.

In an embodiment, the first trained machine learning model estimates a probability of the remote system being in the abnormal state during a time period corresponding to each measurement data unit and the identification of the subset of measurement data units comprises identifying measurement data units corresponding to time periods in which the estimated probability is above a predetermined threshold.

In an embodiment, the first trained machine learning model uses logistic regression to estimate the probabilities. This is explained in detail below with reference to a specific example.

In step S4 of FIG. 3, data representing the measurement data units identified in step S3 are sent over a communications network to the central data processing system 12. In an embodiment, data representing measurement data units that were not identified in step S3 is not transmitted (and may be discarded).

In step S5 of FIG. 3, the central data processing system 12 applies a second trained machine learning model to the data representing the measurement data units received at the central data processing system 12 to detect an abnormal state of the remote system. The detection of the abnormal state may comprise detecting a state indicative of deterioration of the remote system, such that a risk of failure of the remote system 2 in a given time period from the measurement is higher than what would be considered a normal state of the remote system 2. Detection of the abnormal state may thus indicate that the remote system 2 needs attention (e.g. servicing or replacement of parts) to avoid failure. A degree of abnormality may be obtained or various different types of abnormality may be detectable, allowing different types of remedial actions to be initiated (e.g. servicing scheduled for one month's time for a mild abnormal state or immediate action scheduled for a severe or danger posing abnormal state). Alternatively or additionally, the abnormal state may comprise a failed state of the remote system 2, in which normal functionality is hampered or completely absent, such that immediate action is required to restore normal functionality. The processing performed by the central data processing system 12 will typically be performed with a time delay relative to the collection of data by the sensor systems 6 at the remote systems 2 and may therefore be referred to as offline processing herein (as opposed to the processing performed by the local data acquisition units 10, which will typically be performed in real time or near real time to keep up with acquisition of data from the sensor systems 6). Due to the lack of particular restrictions at the central data processing system 12 (e.g. relatively powerful computers, for example many-core workstations, may typically be available), a wide variety of machine learning techniques may be used to implement the second trained machine learning model. The second trained machine learning model may be based on one or more of the following for example: support vector machines; decision tree learning; artificial neural networks; Bayesian networks; and genetic algorithms. However, as described below, the inventors have found that implementing the trained second machine learning model based on either a support vector model (SVM) or a random forest (RF) network classifier model (an example of decision tree learning) works particularly effectively in the context of health monitoring of mechanical systems such as hand-operated pumps.

In an embodiment, the central data processing system 12 generates a web application to allow users to configure the system 20 (e.g. to adjust data transmission choices or protocols) and/or define user alert protocols. In an embodiment, the central data processing system 12 allows a user to tune either or both of the type and the size of data to be transmitted from the remote system 2 to the central data processing system 12. In an embodiment, the central data processing system 12 is configured to be capable of pro-actively requesting more data and/or more detailed data, such as requesting feature vectors or raw accelerometer data rather than novelty scores.

In an embodiment, the central data processing system 12 dynamically adjusts the predetermined threshold used for identifying the subset of measurement data units described above with reference to step S3 of FIG. 3. This provides a simple and efficient mechanism by which the central data processing system 12 can optimize the amount of data flowing from the remote systems 2 to the central data processing system 12. Lowering the predetermined threshold will cause more data to flow from each affected remote system 2 to the central data processing system 12. Raising the predetermined threshold will cause less data to flow from each affected remote system 2 to the central data processing system 12. As demonstrated below with reference to FIGS. 13-15, the central data processing system 12 can be configured to estimate how performance of the second trained machine learning model is expected to vary as a function of the proportion of data that is sent from the remote systems 2 to the central data processing system 12. If the central data processing system 12 determines that it is operating in a relatively flat part of the curve of performance (AUROC) against proportion of data, for example, and/or where there are particular constraints on network capacity or other factors, the central data processing system 12 may raise the predetermined threshold to reduce the proportion of data transmitted from each affected remote system 2. Alternatively or additionally, in an embodiment, the predetermined threshold is lowered by the central data processing system 12 for a given remote system 2 when the second trained machine learning model detects an increase in a probability of an abnormal state of the remote system 2. In this way, the central data processing system 12 can look more carefully at remote systems 2 as soon as their state starts to look suspect, while not using excessive resources at other times. This may increase load on remote systems 2 that are close to failure, potentially leading to earlier failure of batteries or the like, but since such batteries or the like would often need replacing anyway when the failed remote system 2 is visited for repair, this will often not represent a significant downside.

A detailed example specific to monitoring hand-operated pumps is now described.

Unlike in the context of patient-monitoring, there are no standardized labeling protocols for rural infrastructure conditions. Two attributes to classify a state of a hand-operated pump are introduced:

1) Short-Term Water Quantity: a hand-operated pump is either classed as normal (C1) or abnormal (C0). A hand-operated pump is considered normal when water flows from the spout while pumping and abnormal when no water flows from the spout while pumping.

2) Mechanical Performance: ten sub-categories, shown in Table II, are used to identify the mechanical attributes that describe the functionality and physical condition of the hand-operated pump. The data was labeled using notes collected during in-person, contemporaneous observations. This level of labeling is limited in that it allows for only two classes. Certain conditions, like those with average or low flow, are not entirely normal nor entirely abnormal. However, it is believed the proposed labels are adequately descriptive for the purposes of the present demonstration.

TABLE II
Description of the mechanical condition and short-term water
quantity classification labels assigned to each recording.

| Condition Description | Water Flow | Label |
|---|---|---|
| Excellent working condition | High flow | 1 |
| Noisy but working | Average to Low flow | 1 |
| Dry borehole | No flow | 0 |
| Rising main leak | No flow | 0 |
| Broken U-seal | No flow | 0 |
| Worn U-seal | Average to low flow | 1 |
| Handpump body leaking | Average to low flow | 1 |
| Worn bush bearing | Average Flow | 1 |
| Stiff handle | Average Flow | 1 |
| Other | Average to low flow | 1 |

In this example, vibrations of an operating Afridev hand-operated pump were measured via a retrofitted sensor system 6 comprising a consumer grade accelerometer with a sampling frequency of 96 Hz as a sensor element. Each sensor system 6 was housed in a waterproof casing and mounted with tamper-proof bolts inside the handle 4 of the pump at a position close to the pump body, as shown schematically in FIG. 1, without interfering with the range of motion of the handle 4. The accelerometer used in this example was configured to provide measurements relative to three orthogonal axes, with the Y-axis being parallel to a longitudinal axis of the pump handle 4 (as shown in FIG. 1). Examples of a 5 s interval of data from a pump 2 in normal and abnormal conditions are respectively shown in FIGS. 5 and 6.

Following 5 minutes of inactivity, the local data acquisition units 10 switch to a low power state to preserve battery life, restarting after 10 s of continuous motion. For a regularly used hand-operated pump 2, operating nearly constantly for 8 to 12 hours per day, this translates to about 1 gigabyte of data per hand-operated pump 2 per month. All of the hand-operated pumps 2 in the region managed in this example were located in areas with sufficient network coverage to transmit the data via the telecommunications network. However, to preserve battery and cost of data transmission, the data was stored locally on a micro-SD card and downloaded manually for the purposes of this demonstration.

Three data sets were collected from pumps at a site in Kwale, Kenya. The data sets contained high-frequency (96 Hz) three axes accelerometery readings from a local data acquisition units 10 mounted inside the handle 4 of the pumps, as described above. Data from the Y-axis was found to be the most informative. Thus, embodiments are preferably provided in which an accelerometer is attached to the handle and each measurement data unit comprises at least a measurement of acceleration parallel to the longitudinal axis of the handle 4 by the accelerometer.

A significant difference in the spectra of deep and shallow hand-operated pumps was observed, as depicted in FIGS. 7 and 8. However, for demonstration purposes in the present example, analysis was performed primarily on data collected from deep wells, operating at depths greater than 25 m. In many areas, deep hand-operated pumps are typically located at greater elevations where other groundwater sources tend to be sparse, often making them the primary source of drinking water for the nearby communities and households. However, the greater weight of water and hand-operated pump rods being lifted, combined with the increased level of use, leads to more frequent breakdowns of these hand-operated pumps compared to those located at shallower wells. Failures at deeper wells are more labour intensive and time consuming to repair. This, together with their more inaccessible locations, leads to deeper wells often having longer downtimes, making remote condition monitoring and timely repair even more important.

The first data set, Dₘ, represents a general inter-hand-operated pump system consisting of twelve different hand-operated pumps 2 of varying operating depths ranging between 6 m to 53 m, and was included to establish the baseline performance of a general classifier. The second data set, D_{d,1}, represents a deep operating inter-hand-operated pump system consisting of eight different hand-operated pumps 2 operating at depths between 33 m to 54 m. The third data set, D_{d,2}, represents a deep-operating intra-hand-operated pump system of one hand-operated pump operating at 54 m. Although the implementation of a region-wide intra-hand-operated pump system is unfeasible, this data set was selected to investigate the influence of different failure types, while controlling for the hand-operated pump.

The data sets contained recordings from eight different common hand-operated pump failure types. All the data sets were balanced and randomly divided into a training-and-validation set (80%) and a test set (20%).

As a first layer of condition monitoring, the identification of the subset of measurement data units in step S3 of FIG. 3 is desirably implemented using a first machine learning model that is quick to train and fast to classify unknown records, such that it is suitable for applications with limited processing power and bandwidth. In the present example, the first machine learning model is implemented based on logistic regression. In machine learning, logistic regression can be used to model the posterior probability of input variables, X, being associated with a class by fitting a linear model to the feature space. As a linear classification method it is used to categorize the dichotomous dependent variable and predict the probability (0,1) of membership of one class (e.g., True/False) in a two class setting, making it suitable for this lightweight approach.

In the present example, a logistic regression (LR) model was formulated using the sigmoidal hypothesis function, h(xₙ), with a probability that a given example is of class 1:

$$P ( {ℓ}_{n} = 1 \vert {x}_{n} , w ) = h ( {x}_{n} ) = \frac{1}{1 + {e}^{- {w}^{T} {x}_{n}}}$$

where w is a set of weights assigned to each input feature, xₙ. The decision threshold, T, is used to assign a given example to class 1 based on whether the hypothesis function is greater than or less than T. This threshold can be varied to change the size of the data subsets that was subsequently transmitted to the offline classifier (the second trained machine learning model). As the value of T is decreased, the size of the subset s increases, as more of the novelty scores are deemed abnormal.

The LR model was trained using 5-fold cross-validation (CV), where each training set, Dₜ, was randomly subdivided into 5 equal subsets to construct 5 independent training-and-validation sets. The LR regularization parameter, λ, for each independent LR model was optimized by maximizing the area under receiver operator curve (AUROC) on the held-out validation sets.

In this example, the second trained machine learning model implementing the functionality of step S5 of FIG. 3 involved performing heavyweight machine learning processing on the subsets of data flagged (identified) by the lightweight on-board novelty filter (i.e. the first trained machine learning model implementing the functionality of step S3 of FIG. 3 at the remote device 2). In addition to an LR model, support vector machine (SVM) and random forest (RF) classifiers were investigated in detail for implementation of the second trained machine learning model. The novelty filter functionality provided by the first trained machine learning model implementing the functionality of step S3 of FIG. 3 at the remote device 2 is used to ensure that under normal operating conditions the vast majority of data is not transmitted and only when the first trained machine learning model suspects the condition of the remote device 2 is degrading will data be transmitted to the central data processing system 12. This means that in most cases the central data processing system 12 will be receiving data predominantly relating to abnormal conditions of the remote system 2. In an embodiment, to simulate this operating scenario, the second machine learning model is trained using data which contains both normal and abnormal examples and tested using data which only contains examples flagged as abnormal by the first trained machine learning model at the remote device 2. However, in reality, these test examples may contain both normal and abnormal examples given that the first trained machine learning model is likely to misclassify some proportion of data.

For comparison purposes in the present example, the second trained machine learning model was implemented using the LR model described above and tested using the novelty filtered data output from step S3 of FIG. 3.

The second machine learning model was also implemented using an SVM classifier model. The SVM classifier model was trained using the radial basis function, exp(−γ∥x−x′∥²), to project the individual scores from the novelty filter where two classes may be linearly separable. The SVM classifier model was also trained using the 5-fold CV method, using different training and validation sets. The SVM hyperparameters: the kernel bandwidth, γ, and penalty cost factor, C, were optimized using grid search, where γ=2^{a} for a ∈ [−10, −9, . . . 5] and C=2^{b} for b ∈ [−5, −4, . . . 10], by maximizing the sum of all AUCs over all CV folds. The grid search was done independently for each CV fold. Once this was completed, we repeated the process to perform a fine grid search, where aₒₚₜ ∈ [aₒₚₜ−1, aₒₚₜ−0.75, . . . aₒₚₜ+1] and bₒₚₜ ∈ [bₒₚₜ−1, bₒₚₜ−0.75, . . . bₒₚₜ+1]. The refined hyperparameters, γ* and C*, from the fine grid search was used to train the SVM models from the training sets from each of the data sets.

The second machine learning model was also implemented using a Random Forest (RF). The RF classifier model was trained using a random selection of a subset of features, Θₖ, and a random subset of the training data, D(t), to grow each decision tree, T At each node, t, of the tree, the split sₜ=s* to separate the input vector, X, was chosen to minimize the impurity, i(t), in class labels by minimizing the misclassification such that i_{E}(t)=1−max{p_{c}}, where p_{c} is the probability of a class C. The importance of the variable input feature X for predicting the output is based on their weighted impact on decreasing the impurity of that node for all N_{T} trees in the forest:

$$Imp ( {X}_{m} ) = \frac{1}{{N}_{T}} {\sum }_{T} {\sum }_{t ∈ T : v ( {s}_{t} ) = {X}_{m}} \frac{{N}_{t}}{N} Δ i ( {s}_{c} , t )$$

where vₛ is the variable used in split sₜ.

The RF hyperparameters: the number of threes, N_{T}, the number of feature vectors in each decision tree, and the proportion of training data to be bootstrapped were again optimized using a grid search.

Following the analysis above, a condition score was produced in-situ at the remote system 2, Q_{n,i}. Due to the lightweight processing requirement of the local data acquisition unit 10 used in this example, the temporal dependence of the accelerometer observations were not considered at the remote system 2. This was done during postprocessing by aggregating the classifier scores over consecutive examples to varying degrees by applying a moving average (MA) window and increasing the size of the window from 7 s to 27 s. This produced three lightweight condition scores, Q_{n,i}, per data set with i=1 . . . 3 equivalent to [raw on-board score, 7 s MA window score, 27 s MA window score].

The in-situ condition score, Q_{n,1}, was then used to filter the transmitted data such that data summaries sent to the central data processing system 12 contain only abnormal examples, as labeled (identified) by the on-board first trained machine learning model. Finally, condition scores were produced for each of the three offline classifier methods (LR, SVM, RF) using the novelty filtered data.

The ability of the above example implementation to verify CM reliability was assessed using the receiver operating characteristic (ROC) to compare the performance. This metric compares the actual and predicted outputs for each class. The true positive rate (TPR), or sensitivity, of a classifier is defined to be the probability of detection, such that

$$T P R = \frac{\sum TruePositive}{\sum ConditionPos i t i v e} ,$$

and the false positive rate (FPR), or fall-out, is defined to be the probability of a false alarm, such that

$$F P R = \frac{\sum Fal s e P ositive}{\sum Con d i t i o n N e g a t i v e} .$$

Optimizing the area under the ROC (AUC) will maximize hand-operated pump failure detection while simultaneously minimizing false alarms, which can be costly in real-life. In the ideal case, the classifier would be very sensitive (TPR=1) with no false alarms (FPR=0).

For the classification performed by the first trained machine learning model on the remote device 2, the performance of Q_{n,i}, was compared to a baseline control score, Q_{n,lab}, generated in the lab using the same original data but assuming no processing or power constraints as would be experienced on-board the local data acquisition unit 10.

### Performance of First Trained Machine Learning Model (at Remote Device)

Table III shows that the intra-hand-operated pump classifier, {(Q_{n,i}, D_{d,2}} pairs, performs substantially better than the inter-hand-operated pump classifiers, {Q_{n,i}, Dₘ/D_{d,1}} pairs, achieving up to 86.2 percent AUROC compared to 65.7 percent.

TABLE III
Results for field-based, Q_{n, i}, and lab-simulated,
Q_{n, lab}, on-board condition classification scores, given
the mean AUC of 20 iterations (one standard deviation).

| | Dₘ | D_{d, 1} | D_{d, 2} |
|---|---|---|---|
| Q_{n, 1} | 61.32 ± 0.7 | 61.55 ± 0.4 | 68.87 ± 0.3 |
| Q_{n, 2} | 64.76 ± 0.4 | 65.08 ± 0.2 | 77.31 ± 0.2 |
| Q_{n, 3} | 65.26 ± 0.1 | 65.67 ± 0.0 | 86.21 ± 0.0 |
| Q_{n, lab} | 68.89 ± 0.3 | 69.80 ± 0.2 | 88.01 ± 0.2 |

However, the performance of the general inter-hand-operated pump classifier is sufficient to use as a lightweight novelty filter since the large scale implementation of pump-specific classifiers would be too costly and unrealistic to roll-out across entire region-wide rural water supply networks.

In all three cases, the lab generated scores, Q_{n,lab}, outperform those generated by the on-pump classifier, Q_{n,i}, by 7.5 to 12.1 percent. Given the limitations of the embedded system, it was expected that the accuracy of the on-pump classifiers would suffer compared to the lab-simulated results. Due to the lightweight processing requirement of the on-board classifier, the temporal dependence of the accelerometer observations have not been considered. However, post-processing of the ROC scores indicate that the classifier performance improves when temporal correlation is incorporated by aggregating the classifier scores over consecutive examples (to varying degrees as the moving average window size is increased 7 s to 27 s).

This type of post-processing is fairly lightweight and can be easily implemented on-board the hand-operated pump to improve on-pump novelty scores, which will bring it nearly on par with the lab-simulated results.

FIGS. 9-12 compare the receiver operator curve (ROC) scores for the first machine learning model trained using different data subsets for: a) a general classifier trained using Dₘ (FIG. 9); b) a depth-specific inter-handpump classifier trained using D_{d,1} (FIG. 10); c) a broken pump rod in a depth-specific intra-handpump classifier trained using D_{d,2} (FIG. 11); d) a rising main leak in a depth-specific intra-handpump classifier trained using D_{d,2} (FIG. 12). The curves for the general and depth specific inter-hand-operated pump classifiers look almost identical. This is likely a result of the underrepresentation of data from shallow hand-operated pump failures in the training and test sets of the general classifier. Given that deep hand-operated pumps are likely to break more frequently and repairs are more time- and labour-intensive, the need for such classifiers are more important for deep operating hand-operated pumps.

The case studies shown in FIGS. 9-12 demonstrate two key findings: (i) general on-board classifiers may perform sufficiently well as not to necessitate the need for depth-specific classifiers, as shown in FIGS. 9 and 10; and (ii) it may be possible to identify specific failure types if the system has a priori knowledge of the hand-operated pump operating depth. However, certain extreme failure types that are physically located closer to the sensor system 6, like a broken hand-operated pump rod shown in FIG. 11, are easier to detect than less severe failures located further away from the sensor system 6, like a leak in the rising main 43 m down the borehole shown in FIG. 12. It may also confirm the limitations in the classification labels of the data, such as labeling low water flow caused by a leak in the rising main as abnormal when in reality it is neither entirely normal nor entirely abnormal but rather indicative of an imminent failure event than a failure in itself.

### Performance of Second Trained Machine Learning Model (at Central Data Processing System)

Table IV shows that in all three cases the LR classifier is sufficiently lightweight in that it reaches the optimum classification accuracy by using only 6 to 15 percent of the flagged data (the identified subset of measurement data units) from the first trained machine learning model, compared to 89 to 98 percent required by the RF classifier and 97 to 100 percent for the SVM. In all three cases, the RF classifier outperforms the LR and SVM classifiers.

As before, the LR classifier shows little difference in performance between a general, Dₘ, or depth-specific, D_{d,1}, inter-hand-operated pump data set. The RF classifier does marginally better for depth-specific, D_{d,1}, data set. Both the LR and RF classifier performance improve.

TABLE IV
Results for lab-based offline condition classification
of on-pump processed data, given the mean AUC
of 20 iterations (one standard deviation).

| | | Dₘ | D_{d, 1} | D_{d, 2} |
|---|---|---|---|---|
| Offline | LR | 59.6 ± 0.1 | 60.0 ± 0.0 | 79.4 ± 0.0 |
| scores at | RF | 71.8 ± 2.5 | 70.9 ± 2.0 | 86.8 ± 1.4 |
| 100% data | SVM | 60.8 ± 0.2 | 65.2 ± 0.0 | 75.0 ± 0.0 |
| Max | LR | 60.9 ± 1.8 | 60.7 ± 2.3 | 80.8 ± 2.7 |
| offline | | at 8% | at 6% | at 15% |
| scores | RF | 73.0 ± 1.7 | 72.1 ± 2.3 | 87.5 ± 1.5 |
| | | at 89% | at 98% | at 91% |
| | SVM | 60.8 ± 0.6 | 65.3 ± 0.5 | 75.0 ± 0 |
| | | at 99% | at 97% | at 100% |

Unlike the RF and LR classifiers, the SVM classifier performance benefits more from the depth-specific data set, D_{d,1}, than the general data set, Dₘ, since two-class SVMs are trained to have a low misclassification rate. The SVM classifier performance is likely to increase as we continue to collect more depth-specific data. This is suggested by the significant reduction in variance for the SVM classifier as the proportion of data is increased.

FIGS. 13-15 compare the AUROC scores for the three types of classifiers trained using the three different proposed data sets. The figures show AUROC comparison of the offline classifier using novelty filtered data subsets for: a general classifier trained using Dₘ (FIG. 13); a depth-specific inter-handpump classifier trained using D_{d,1} (FIG. 14); and a depth-specific intra-handpump classifier trained using D_{d,2} (FIG. 15). In all three sets, the offline LR classifiers (implemented by the central data processing system 12) benefits the least from the addition of an increase in the subset used for testing. However, the standard deviation in the predictive accuracy of the LR model does reduce as the test set size increased.

Conversely, however, in all three cases the RF classifier achieves the highest overall accuracy, with relatively little data, and benefits minimally from more data both in improving prediction accuracy or decreasing prediction variance.

Overall, more heavyweight offline machine learning methods (as performed by the second trained machine learning model as opposed to the first trained machine learning model) offer a 10 percent improvement from the raw on-pump generated condition scores, Q_{n,i}, in the above example. The three cases show that there is a trade-off between accuracy and specificity. Whilst the RF classifier may offer a higher overall prediction accuracy, both LR and SVM can dramatically reduce the variability in predictions as the proportion of data supplied is increased. This is an important trade-off to note since it may have a direct impact on operational cost.

### Improving Real-Time Performance

To ensure the system is suitable for real-time implementation within the constraints of the limited resources, such as battery life and data transmission, two additional design factors were considered that facilitate leaner implementation with minimal effect on overall system performance.

For optimizing the run time cost model of operating such a large-scale distributed system, the impact was considered of potentially distributed run time plans and the machine learning characteristics of each classifier as a direct trade-off of its overall prediction accuracy.

1) Prediction Run Time: Time implementation of complex, region-wide monitoring systems should aim to optimize machine learning approaches by being sensitive to memory use and parallelism. FIGS. 16 and 17 show comparison of the classifier prediction run times as the proportion of data transmitted from the remote system 2 by the first trained machine learning model varies for: inter-hand-pump (FIG. 16), and intra-hand-pump (FIG. 17) condition monitoring systems. In both cases, the prediction time for the LR and RF classifier remains constant as the proportion of data increases while SVM prediction time increases linearly. As the most lightweight method, the LR classifier shows the fastest run time, irrespective of the system type.

2) Number of Features: FIGS. 18 and 19 show comparison of the classifier performance for varying number of features on two deep well data sets for: inter-hand-pump (FIG. 18), and intra-hand-pump (FIG. 19) condition monitoring systems. In both cases, the intra- and inter-hand-operated pump condition monitoring systems gain very little predictive accuracy from using more than 8 or 10 features, respectively. This suggests that the cost of data transmission during implementation can be reduced by nearly half by reducing the size of the data packages required by the offline system. The trade-offs between the different classifiers and respective prediction times along with the number of features transmitted as part of the measurement data units transmitted from the remote systems 2 to the central data processing system 12 are thus important design considerations for efficient implementation of the final distributed system without sacrificing the performance of the system.

In summary, existing systems for monitoring remote systems are not suitable for monitoring rural infrastructure that often operate in harsh environments and with constraints on data-transmission and battery life. In the present disclosure, an appropriate set of labels is described that can be used as the basis for monitoring the condition of hand-operated pumps (Table II). Embodiments are described in which low-cost, lightweight machine learning methods (the first trained machine learning models) are implemented on-board monitored pumps with minimum bandwidth and battery requirements to apply novelty filtering (see FIGS. 9-12 and Table III). Furthermore, incorporating more heavyweight condition monitoring methods on a cloud-based platform (i.e. the second trained machine learning model implemented at the central data processing system 12) have been shown to increase the system's overall positive predictive value by 10 percent when the identified subsets of data from the remote pump is transmitted (see FIGS. 13-15 and Table IV).

The inventors found that distributed inference using logistic regression (LR) to implement the first machine learning model (on-board the remote system 2) followed by random forests (RF) to implement the second machine learning model (at the central data processing system 12) provided the best performing monitoring for rural infrastructure while optimizing limited resources. We found that the combination of LR and RF provides the optimal prediction run time and can both be successfully implemented with less than half the number of features to be transmitted from the remote system to the central data processing system.

The embodiments described above focus on application of the system to monitoring hand-operated pumps, but the same overall architecture can be applied to other rural infrastructure types, such as off-grid home solar systems or agriculture monitoring systems.

1. A method of monitoring a remote system, comprising:
obtaining a plurality of measurement data units, each measurement data unit representing a time series of measurements made by a sensor system at the remote system;
using a first trained machine learning model to identify a subset of the measurement data units that have a higher average probability of corresponding to an abnormal state of the remote system than the other measurement data units;
sending data representing the identified measurement data units over a communications network to a central data processing system; and
detecting an abnormal state of the remote system by using a second trained machine learning model at the central data processing system to process the data representing the identified measurement data units.

2. The method of claim 1, wherein the first trained machine learning model estimates a probability of the remote system being in the abnormal state during a time period corresponding to each measurement data unit and the identification of the subset of measurement data units comprises identifying measurement data units corresponding to time periods in which the estimated probability is above a predetermined threshold.

3. The method of claim 2, wherein the predetermined threshold is dynamically adjusted by the central data processing system via the communications network.

4. The method of claim 3, wherein the predetermined threshold is lowered by the central data processing system in response to the second trained machine learning model detecting an increase in a probability of an abnormal state of the remote system.

5. The method of any of claim 2, wherein the first trained machine learning model uses logistic regression to estimate the probabilities.

6. The method of claim 1, wherein the remote system comprises a mechanical apparatus.

7. The method of claim 6, wherein the sensor system comprises an accelerometer.

8. The method of claim 7, wherein the accelerometer is attached to a moving part of the mechanical apparatus.

9. The method of claim 6, wherein the mechanical apparatus is a hand-operated water pump comprising a movable handle for hand-operating the water pump.

10. The method of claim 9, wherein the sensor system comprises an accelerometer configured to measure a component of acceleration of the handle parallel to a longitudinal axis of the handle.

11. The method of claim 1, further comprising pre-processing the measurement data units before the first trained machine learning model identifies the subset of the measurement data units.

12. The method of claim 11, wherein the pre-processing comprises determining a period of a largest periodic component of the times series of measurements in each measurement data unit, and the pre-processing comprises removing measurement data units in which a period of the determined largest periodic component is below a predetermined threshold period.

13. The method of claim 12, wherein the remote system comprises a hand-operated water pump and the predetermined threshold period equals 0.5 s.

14. The method of claim 11, wherein the pre-processing comprises applying a high pass filter to each measurement data unit.

15. The method of claim 11, wherein the pre-processing comprises transforming the measurement data units to represent the time series of measurements in the frequency domain.

16. The method of claim 1, wherein the second trained machine learning model comprises a support vector machine or a random forest classifier model.

17. The method of claim 1, wherein the remote system comprises an electrical infrastructure system.

18. The method of claim 1, wherein the remote system comprises a biological system.

19. The method of claim 18, wherein the biological system is a human or animal and the sensor system is configured to measure one or more parameters relevant to a state of health of the human or animal.

20. A system for monitoring a remote system, comprising:
a local data acquisition unit comprising a sensor system and a local data processing unit; and
a central data processing system;
wherein:
the local data processing unit is configured to:
obtain a plurality of measurement data units, each measurement data unit representing a time series of measurements made by the sensor system at the remote system;
use a first trained machine learning model to identify a subset of the measurement data units that have a higher average probability of corresponding to an abnormal state of the remote system than the other measurement data units; and
send data representing the identified measurement data units over a communications network to the central data processing system; and
the central data processing system is configured to:
detect an abnormal state of the remote system by using a second trained machine learning model to process the data representing the identified measurement data units received from the local data acquisition unit.
